(19) United States
(12) Patent Application Publication
(10) Pub. No.: US 2014/0378738 A1
(43) Pub. Date: Dec. 25, 2014

(54) IRRADIATION SYSTEM

(71) Applicant: SIEMENS AKTIENGESELLSCHAFT, MÜNCHEN (DE)

(72) Inventor: BELA VAJKO, ERLANGEN (DE)

(21) Appl. No.: 14/299,091

(22) Filed: Jun. 9, 2014

(30) Foreign Application Priority Data
Jun. 25, 2013 (DE) 102013212088.5

(51) Int. Cl. A61N 5/10; A61B 18/18; A61B 5/055

(52) U.S. Cl. CPC A61N 5/1049; A61B 5/055; A61B 18/18; A61N 2005/1055
USPC 600/2

(57) ABSTRACT

An irradiation system for simultaneous application of radiation therapy and hyperthermia is provided. The irradiation system has a radiation source and a magnetic resonance tomography apparatus comprising a controller, a plurality of local coils for arrangement on the body of a patient, and a plurality of transmission devices. The transmission devices are configured to supply the local coils with radiofrequency signals independently of one another. In this case, the controller is configured to actuate the transmission devices in such a way that a predetermined region in the body of the patient is heated to a predetermined temperature by means of the radiofrequency signals emitted by the local coils. The radiation source is configured to irradiate a target in the predetermined region.

### CROSS-REFERENCE TO RELATED APPLICATIONS

This application claims priority to DE Application No. 102013212088.5, having a filing date of Jun. 25, 2013, the entire contents of which are hereby incorporated by reference.

### FIELD OF TECHNOLOGY

The following relates to an irradiation system for simultaneous application of radiation therapy and hyperthermia. The irradiation system comprises a radiation source for irradiating a patient and a magnetic resonance tomography apparatus. The magnetic resonance tomography apparatus furthermore comprises a controller, a plurality of local coils for arrangement on the patient, and a plurality of transmission devices, the transmission devices being configured to supply the local coils with radiofrequency signals independently of one another.

### BACKGROUND

For the purpose of treating tumors, it has long been known to irradiate said tumors with high-energy electromagnetic waves from the x-ray or gamma spectrum or else with particle beams in order to damage tumor cells and cause them to die.

It is known from the publication titled “Simultaneous delivery of electron beam therapy and ultrasound hyperthermia using scanning reflectors” (International Journal of Radiation Oncology Biology Physics, Volume 31, Issue 4, pages 893-904, E. Moros, W. Straube, E. Klein, M. Yousaf, R. Myerson) to perform an irradiation treatment by means of an electron beam concurrently with hyperthermia by means of ultrasound (high-intensity focus ultrasound (HIFU)), thereby intensifying the effect of the electron beam on the tumor cells.

Systems are also known in which a magnetic resonance tomography apparatus is used in order to support and control an irradiation device while the latter is being aligned onto the target that is to be irradiated (e.g. systems from ViewRay Inc.)

In magnetic resonance measurements, the interaction of magnetic moments of atomic nuclei, the nuclear spins, with an external magnetic field is investigated. The nuclear spins align themselves in the external magnetic field and precess at a Larmor frequency which is dependent on the value of the magnetic moment of the atomic nucleus and the external magnetic field upon excitation by means of an external alternating electromagnetic field about the axis of the alignment in the magnetic field. In so doing, the atomic nuclei generate an alternating electromagnetic field at the Larmor frequency. The alternating electromagnetic field radiated in in order to excite the spins leads in the process to a heating of the object under examination due to absorbed power.

The use of ever-stronger magnetic fields of up to 3 T and more in magnetic resonance technology has also produced an increase in the frequency of the radiofrequency excitation signals, resulting in turn in a squared increase in the specific absorption rate (SAR). Meanwhile, the measurement time can be limited by means of the SAR, as also can the maximum permissible heating of the body of the patient by means of the radiofrequency signals.

Monitoring of the local absorption rate during parallel excitation by means of a plurality of coils is known from the publication titled “Local specific absorption rate control for parallel transmission by virtual observation points”, Eichfelder G., Gebhardt M., Department of Mathematics, University of Erlangen-Nuremberg, Erlangen, Germany, Magn. Reson. Med. 2011 November; 66(5):1468-76. doi 10.1002/mrm.22927. Epub 2011 May 20.

### SUMMARY

An aspect relates to providing a device and a method which improve the effectiveness of an irradiation with minimum investment of effort and resources. Another aspect relates to an irradiation system and a method for operating the irradiation system.

The irradiation system for simultaneous application of radiation therapy and hyperthermia comprises a radiation source for irradiating a patient and a magnetic resonance tomography apparatus. The magnetic resonance tomography apparatus comprises a controller, a plurality of local coils for arrangement on the patient, and a plurality of transmission devices, the transmission devices being configured to supply the local coils with radiofrequency signals independently of one another. In this arrangement, the controller can be configured to actuate the transmission devices in such a way that a predetermined region in the body of a patient is heated by a predetermined temperature by means of the radiofrequency signals emitted by the local coils, while the radiation source can be configured to irradiate a target in the predetermined region.

The independent local coils and transmission devices permit the controller to transmit energy by means of electromagnetic waves into the patient's body so as to heat the latter at predetermined points. The heat applied to the target that is to be irradiated reinforces the biological effectiveness of the irradiation. Furthermore, the magnetic resonance tomography apparatus also enables an imaging visualization such that the alignment of the irradiation onto the target that is to be irradiated, for example a tumor, can be optimized.

In a possible embodiment, the irradiation system is configured either to actuate the transmission device by means of a first control signal which is configured to heat a predetermined region in the body of the patient to the predetermined temperature by means of the transmission devices and the local coils or to actuate the transmission device by means of a second control signal which is configured to generate an imaging visualization by means of the magnetic resonance tomography apparatus. This can make it possible to optimize the radiofrequency pulses for the respective purpose, i.e. imaging or thermal effect.

In a possible embodiment, the controller of the irradiation system is configured to actuate the transmission device by means of the first control signal when the radiation source emits no radiation onto the patient.

Accordingly, it is possible to monitor the position of the irradiation target by means of magnetic resonance tomography imaging during an irradiation phase in which the patient is irradiated by the radiation source, while in the irradiation pauses the transmission devices can output optimized radiofrequency pulses in order to heat the region that is to be irradiated.

The method for operating the irradiation system may share aspects of the system described above.

In a possible embodiment, the magnetic resonance tomography apparatus of the irradiation system is furthermore configured to register an increase in temperature in the predetermined region by means of a magnetic resonance measurement. The output amplitude, the phase coherence of the precession of the nuclear spins and also the decline in the excitation can change as a function of the temperature of a sample in which the atomic nuclei are located. By comparing two measurements, it is possible to determine a change in temperature of the sample between the two measurements by means of the magnetic resonance tomography apparatus.

In an exemplary embodiment, the predetermined temperature has a value between normal body temperature and 42 degrees Celsius or the predetermined temperature is equal to 42 degrees Celsius. Heating the body tissue to 42 degrees Celsius during the irradiation phase can ensure an optimal effect of the irradiation in the heated tissue, while non-irradiated tissue is not damaged by the increase in temperature.

In an exemplary embodiment of the irradiation system, each of the plurality of local coils is configured in such a way that the local coils are not mutually influenced by their signals. Because the antennas do not mutually affect one another, or do so only to a minor extent, the signals injected into the local coils by the transmission devices can be set independently of one another by the controller, which simplifies the determining of the signals that are to be set.

In a possible embodiment of the irradiation system, the controller and the transmission devices are configured to provide the radiofrequency signals of the individual local coils with a predetermined phase angle in each case. Setting the phase angle enables, by overlaying the electromagnetic fields transmitted by the individual local coils, a resulting overlay field to be generated with a directional effect and a predefined field distribution.

In an exemplary embodiment of the irradiation system, the controller has a determination unit, the determination unit being configured to determine control signals for the transmission devices on the basis of a predetermined temperature distribution in the patient by means of which the predetermined temperature distribution in the patient can substantially be achieved. The determination unit enables the desired temperature distribution to be predefined, whereupon the determined control signals which are output by the controller to the transmission devices lead via the local coils to alternating electromagnetic fields in the tissue which bring about the temperature distribution in the body of the patient. Essentially, this means that the temperatures, limited by the physical factors such as thermal diffusion and wavelength, can map specific profiles only within the natural limits, with the result that e.g. a maximum temperature gradient cannot be exceeded. What is also expressed thereby in particular is that the temperature distribution does not exceed certain safety criteria, such as a maximum temperature.

In a possible embodiment of the irradiation system, the controller is configured to actuate the transmission device in such a way that a deviation between the predetermined temperature distribution and the measured increase in temperature is reduced.

Accordingly, embodiments of the irradiation system can enable a possible deviation between the first control signals determined by the determination unit and the thus achieved heating from the desired temperature distribution to be determined and corrected. In this way, the deviation can be minimized and at the same time the observation of safety limit values can be guaranteed by taking into account the true temperature distribution.

### BRIEF DESCRIPTION

Some of the embodiments will be described in detail, with reference to the following figures, wherein like designations denote like members, wherein:

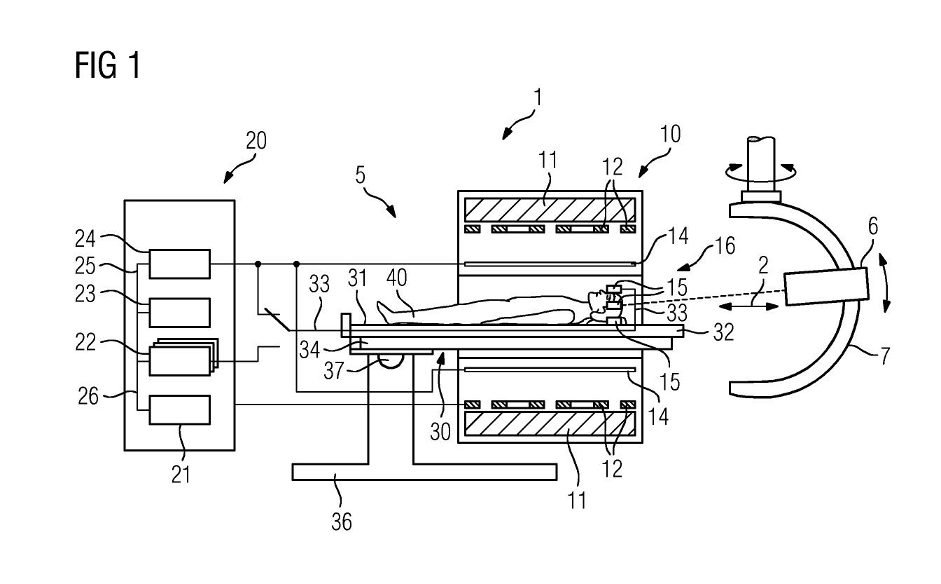

FIG. 1 shows a schematic representation of an embodiment of an irradiation system;

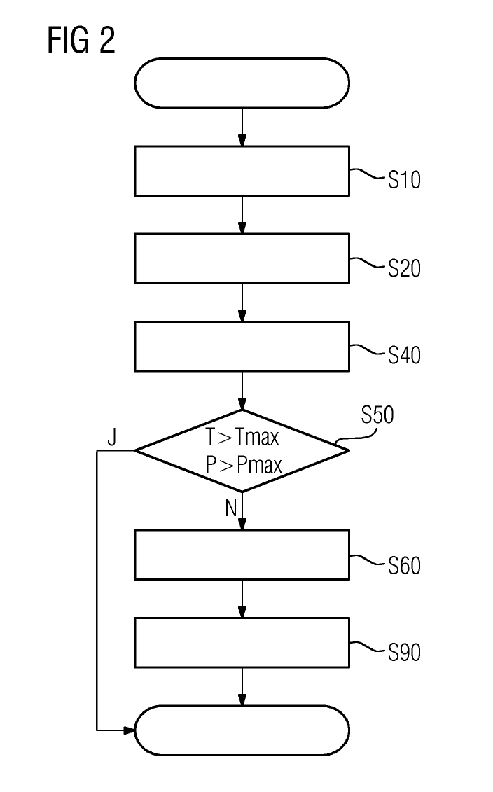

FIG. 2 is a flowchart for an embodiment of a method; and

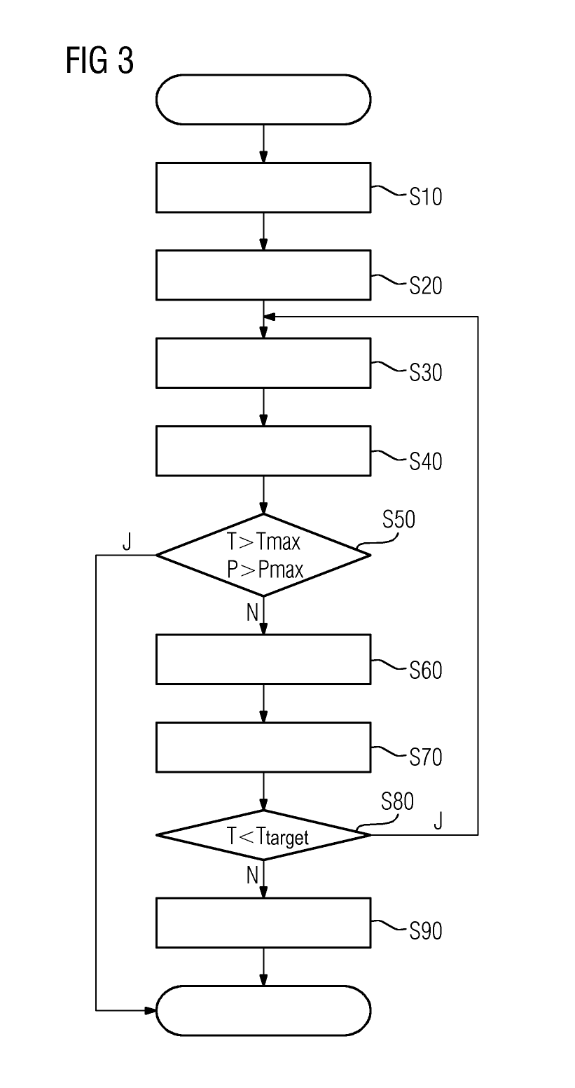

FIG. 3 is a flowchart for an embodiment of a method.

### DETAILED DESCRIPTION

FIG. 1 shows a schematic representation of an irradiation system 1 for performing the method. The irradiation system has a magnetic resonance tomography apparatus 5 for acquiring an image of a region of a patient's body. The irradiation system additionally has a radiation source 6.

The magnetic resonance tomography apparatus 5 has a magnet unit 10 having a field magnet 11 which generates a static magnetic field B0 for aligning nuclear spins of samples or of a patient 40 in a sample volume. The sample volume is arranged in a tunnel 16 which extends in a longitudinal direction 2 through the magnet unit 10. Typically, the field magnet 11 is a superconducting magnet which is able to provide magnetic fields having a magnetic flux density of up to 3 T, even more in the latest devices. For lower field strengths, however, it is also possible to utilize permanent magnets or electromagnets having normally conductive coils.

In addition, the magnet unit 10 has gradient coils 12 which are configured to overlay the magnetic field B0 with variable magnetic fields in three spatial directions for the purpose of spatially differentiating the acquired imaging regions in the sample volume. The gradient coils 12 are typically coils made from normally conductive wires which can generate fields orthogonal to one another in the sample volume.

The magnet unit 10 additionally includes a whole-body coil 14 and local coils 15. The whole-body coil 14 is used as a transmit coil when a maximally homogeneous electromagnetic excitation field is to be generated over a large volume. The local coils 15 can be arranged as a two-dimensional or three-dimensional array and cover the entire body of the patient 40 or alternatively only the part that is to be irradiated. The local coils 15 serve among other things as transmit coils in order to radiate electromagnetic waves into a substantially spatially limited volume of the body in each case. In such an arrangement, the local coils 15 can for example be circular or polygonal coils which partially overlap one another. What is achieved in this way is that the fields of adjacent coils partially overlay one another in the same and in the opposite direction such that neighboring coils do not reciprocally influence one another to a substantial degree.

The overlapping arrangement of the transmit coils 15 also ensures that an alternating electromagnetic field can be radiated into the whole of the region covered by the coils that is to be irradiated.

A magnetic resonance signal which is excited by means of the alternating electromagnetic field of the transmit coils 15 and the static magnetic field B0 in the patient can be received either once again by the transmit coils 15 or else by a separate whole-body coil 14 which is able to receive signals from the entire region under examination.

In embodiments of the irradiation system, it is conceivable to dispense with the whole-body coil 14 and simply use the local coils 15 both for transmitting and for receiving radiofrequency electromagnetic waves.

A control unit 20 supplies the magnet unit 10 with the different signals for the gradient coils 12 and the whole-body coil 14 or the local coils 15 and evaluates the received signals. Thus, the control unit 20 has a gradient controller 21 which is configured for supplying the gradient coils 12 via supply lines with variable currents which provide the desired gradient fields in the sample volume in a time-coordinated manner.

In addition, the control unit 20 has a plurality of transmission devices 22 which are configured for generating for each transmit coil 15 a radiofrequency pulse having a predefined time characteristic, amplitude, phase and spectral power distribution for exciting a magnetic resonance of the nuclear spins in the patient 40. Pulse powers in the kilowatt range can be achieved in this case.

The receive unit 24 is configured to evaluate radiofrequency signals received from the whole-body coil 14 (or a transmit coil 15) and supplied via a signal line 33 to the receive unit 24 in respect of amplitude and phase. In this case, such signals are in particular radiofrequency signals which transmit nuclear spins in the patient 40 in response to the excitation by means of a radiofrequency pulse in the magnetic field B0 or in a magnetic field resulting from an overlaying of B0 and gradient fields.

Furthermore, the control unit 20 has a controller 23 which is configured to perform the time coordination of the activities of the gradient controller 21 and the transmission devices 22 for the purpose of image acquisition by means of magnetic resonance tomography. Toward that end, the controller 23 is connected to the other units 21, 22, 24 via a signal bus 25 and engages in signal exchange therewith. The controller 23 is configured to receive and process signals evaluated by the receive unit 24 from the patient 40 or to specify pulse and signal shapes to the gradient controller 22 and the transmission devices 22 and to perform the time coordination.

The patient 40 is arranged on a patient couch 30. Patient couches of said type are already known from magnetic resonance tomography. The patient couch 30 has a first support 36 which is arranged under a first end 31 of the patient couch 30. To ensure that the support 36 can hold the patient couch 30 in a horizontal position, it typically has a pedestal which extends along the patient couch 30. In order to move the patient couch 30, the pedestal can also have means for moving, such as rollers. Except for the support 36 at the first end 31, no structural element is arranged between the floor and the patient couch, so the patient couch can be introduced as far as the first end 31 into the tunnel 16 of the field magnet 11. In FIG. 1, linear rail systems 34 are depicted which movably connect the support 36 to the patient couch 30 so that the patient couch 30 can be displaced along the longitudinal direction 2. For that purpose, the linear rail system has a drive 37 which enables the patient couch 30 to move in the longitudinal direction 2 under the control of an operator or alternatively the controller 23, which means that it is also possible to examine regions of the patient's body which have greater dimensions than the sample volume in the tunnel 16.

In order to generate a predetermined temperature in a predetermined region in the body of the patient, the controller 23 actuates the transmission devices 22 by means of first control signals so that said devices output radiofrequency output signals to the transmit coils 15. In this case, the controller 23 is able to influence the frequency and/or amplitude of the signals and/or their phase angle relative to one another.

In this regard, it is conceivable in an embodiment for specific output signal shapes to be stored in a predetermined manner in the controller for specific regions that are to be heated. In this case, the change in temperature could scale in line with the power of the sum of the signals. In the simplest case, it would also be conceivable for the volume to be predefined solely based on the actuation of a single transmit coil 15 and for the region of the body of the patient 40 disposed under the respective coil to be heated.

In another embodiment, the controller 23 could have a determination unit 26 which is configured to determine the output signal and the associated first control signal for the transmission devices 22 from a predetermined temperature distribution for the body of the patient 40 with reference to a model for the absorption of the electromagnetic waves in the body and for the overlaying of the electromagnetic waves of the individual transmit coils 15 in each case for each transmit coil 15. The controller 23 then outputs the corresponding first control signals in a time-coordinated manner to the corresponding transmission devices 22, with the result that the latter generate the appropriate output signals for transmit coils 15 and said signals are radiated into the body of the patient 40 in order to generate the desired temperature distribution there.

In this case, it is possible in an exemplary embodiment of the irradiation system for the determination unit 26 to take into account safety limit values so that for example a temperature of 42 degrees Celsius will not be exceeded in any region of the body of the patient or that a maximum energy will be radiated in during a time period.

In an exemplary embodiment of the irradiation system, it is also conceivable for the magnetic resonance tomography apparatus 5 to determine the temperature distribution in the body of the patient 40 by means of two measurements recorded in succession. The output amplitude, the phase coherence of the precession of the nuclear spins and also the decline in the nuclear spin excitation can change as a function of the temperature of a sample in which the atomic nuclei are located. By comparing two measurements it is possible by means of the magnetic resonance tomography apparatus 5 to determine a change in temperature of the sample between the two measurements.

It is then conceivable in this case that in determining the first control signals for the transmission devices 22 the determination unit 26 not only uses a model for the body and its absorption characteristics, but also takes into account the actual temperature distribution from the measurement in order to correct or even replace the model calculation. This process can also be optimized iteratively over a plurality of sequential excitation pulses.

In an exemplary embodiment, it is also conceivable that during the determination of the first control signals account is also taken of the increase in temperature due to radiofrequency pulses which are required for an imaging of the body of the patient in order to control the alignment of a radiation source 6 and are generated in the transmission devices 22 by means of the second control signals.

In embodiments of an irradiation system, the radiation source 6 is arranged in such a way that there is free access to the region in the body of the patient 40 that is to be irradiated. By this is to be understood, inter alia, that no part of the magnet unit 10 will be located in a beam path of the radiation source 6 and attenuate or scatter the radiation to any significant degree. This relates in particular to metallic parts such as magnet coils, cryostat or antennas. In the magnetic resonance tomography apparatus 5 shown, this is possible above all via the openings of the tunnel 16 in the longitudinal direction 2. However, a split field magnet 11 would also be conceivable, thereby allowing the patient 40 to be accessed from the side, through which side access an irradiation can be performed. In order to enable alignment of the radiation source, the latter is movably mounted on a retaining fixture, for example a C-arm 7. In order to allow better access for the irradiation, an open magnet unit is conceivable in a further embodiment, said magnet unit having a magnet in a horseshoe shape for example. In this case, however, the possible magnetic field strengths are reduced and amount for example only to 1.5 T or less.

X-ray tubes or also gamma sources which emit high-energy photons can be used as radiation source 6. Also conceivable, however, are particle beam sources such as linear accelerators which emit electrons, protons or heavy ions at high energy levels. With charged particle beams, it might be necessary in this case to allow for the deflection of the particles due to the magnetic fields of the field magnet 11 and the gradient coils 12 or to choose a beam direction parallel to the magnetic field lines, for example in the longitudinal direction 2.

In this case, a mounting assembly 7 enables the radiation source 6 to be oriented in its alignment in relation to the patient by means of the control unit 20 in such a way that the radiation source 6 is for example targeted onto a tumor whose position has been determined by means of magnetic resonance tomography and whose environment has been heated to 42 degrees Celsius by means of the alternating electromagnetic field emitted by the transmit coils 15. With large radiation sources, it is conversely also conceivable for the patient 40 to be aligned relative to the radiation source 6 by means of the magnetic resonance tomography apparatus 5.

FIG. 2 shows a flowchart of a method for operating the irradiation system according to the invention.

At step S10, a desired temperature profile for the region of the body of the patient 40 that is to be irradiated is predefined to the controller 23. This can be accomplished for example by specification of a location in the body, a maximum temperature and a temperature gradient by an operator at the control unit 20, which for example has a graphical input interface. It is, however, also conceivable to import a prepared file containing a three-dimensional model.

At a step S20, a magnetic resonance tomography image of the patient 40 is acquired in the tunnel 16. In this case, the transmission devices 22 are actuated by means of second control signals for imaging by means of magnetic resonance tomography. From the magnetic resonance tomography image, the control unit 20 then determines the precise position of the region that is to be irradiated or an operator marks said position on a graphical user interface, for example.

At step S40, the controller 23 determines, from the predetermined temperature profile, the first control signals for the transmission devices 22 which are necessary for achieving the increase in temperature.

The first control signals can be taken for example from a database which holds different data records available for the different positions of the predetermined region. In this case, the power can scale linearly in line with the desired increase in temperature.

It is, however, also conceivable for a determination unit 26 to determine suitable first control signals for the transmit coils 15 by means of a model for the absorption of the electromagnetic waves in the body and a model for the overlaying of the electromagnetic waves emitted by the individual transmit coils 15.

The first control signals at step S40 are different in this case from second control signals at step S20 that are intended for imaging. Depending on the type of imaging, second control signals having a different frequency, amplitude and/or phase angle are required.

In both cases, the determined values are checked at step S50 for a possible exceeding of safety limit values, for example a maximum temperature of 42 degrees Celsius or a maximum radiated energy. In some embodiments, only if the limit values are not exceeded, are the first control signals output, as described hereinbelow.

At step S60, the first control signals are output to the transmission devices 22, which generate the determined radiofrequency signals and transmit same via the transmit coils 15.

At step S90, the radiation source is aligned onto the target whose position was determined in step S20 and the target is irradiated.

FIG. 3 shows a further embodiment of a method. Steps labeled with the same reference numerals are identical to the corresponding steps from FIG. 2.

As with reference to FIG. 2, a desired temperature profile is acquired at step S10, and at step S20 an image of the patient or of the region that is to be irradiated is acquired by means of magnetic resonance tomography.

At step S30, a further magnetic resonance measurement is performed in order to determine a change in temperature.

At step S40, the first control signals are determined, the temperature values measured at step S30 during the determining of the first control signals being taken into account in the model.

As described with reference to FIG. 2, the determined first control signals are checked at step S50, and at step S60 the first control signals are output to the transmission devices 22, the alternating electromagnetic field is transmitted via the transmit coils 15 and the predetermined region of the body of the patient 40 is heated.

At step S70, a further temperature measurement of the body of the patient 40 or of the region is performed by means of magnetic resonance tomography.

At step S80, the controller 23 compares the measured values of the increase in temperature with the values calculated in the model. If the desired increase in temperature has not yet been reached, steps S40 to S70 are repeated until the desired temperature distribution has been achieved.

It is furthermore conceivable to repeat the steps from step S20 after step S90 in order for example to detect and take into account motions of individual organs or movements of the patient as a whole, for example by correctively adjusting the radiation source 6 to track the change in position. In a possible embodiment, step S20 is repeated in this case during the entire time in which the target is irradiated. In this way, the position of the target is continuously monitored and if necessary the irradiation interrupted in the event of unduly large deviations or the alignment of the radiation source 6 onto the target correctively adjusted.

In an exemplary embodiment, it is conceivable for the measurement of the change in temperature to be performed in steps S30 and/or S70 by means of a fast method for measuring a change in temperature by means of magnetic resonance tomography, as is described in the German patent application with the application number 102012221463 not yet published at the time of the filing of the present invention.

This method for measuring a change in temperature comprises step a) of exciting nuclear spins in a sample volume by means of a radiofrequency pulse in a magnetic field, step b) of acquiring and storing a first projection of an integral of a magnetic resonance signal in the sample volume over a first n-dimensional space onto a first m-dimensional space by means of a multi-echo sequence, where n+m=3, and a first projection of an integral of a magnetic resonance signal in the sample volume over a second n-dimensional space onto a second m-dimensional space by means of a multi-echo sequence, where the first and the second m-dimensional space are not parallel to each other and where n+m=3. Embodiments of the method further comprises step c) of repeating step b), wherein a second projection is acquired and stored in each case, and step d) of forming a difference between the first projection and second projection over the first n-dimensional space and forming a difference between the first projection and second projection over the second n-dimensional space in order to determine a change in temperature.

The method for measuring a change in temperature entails acquiring a projection onto two m-dimensional spaces which are not parallel to each other. In this case the term space is to be understood in the mathematical sense, not only as a Euclidean space having three coordinate axes standing perpendicular to one another. A one-dimensional space in this context is a line or straight line, a two-dimensional space an area or plane. The sum of the natural numbers n and m is 3 in each case and corresponds to the number of dimensions of the Euclidean space. It is therefore possible to register a spatial position of a region of changed temperature by means of only two projections, instead of having to scan the entire sample volume spatially in slices. In this, way transient temperature peaks can also be detected, whereas artefacts which have experienced no change in the short time period therebetween are masked out.

Embodiments of the method for operating an irradiation system as well as an irradiation system suitable for performing the method for measuring a change in temperature permit both the temperature of the region to be changed by means of radiofrequency radiation and the change in the temperature to be monitored by means of a measurement in the pauses between individual irradiation intervals, without noticeably lengthening the overall process. Since the applied temperatures themselves are not directly harmful to the tissue, it is sufficient in particular if the occurrence of a dangerous heat buildup can be precluded by means of a rapidly achievable two-dimensional projection of the temperature distribution.

Although the present disclosure has been illustrated and described in more detail on the basis of the preferred exemplary embodiment, it is not limited by the disclosed examples and other variations can be derived herefrom by the person skilled in the art without leaving the scope of protection of the invention.

1. An irradiation system for simultaneous application of radiation therapy and hyperthermia,
wherein the irradiation system comprises:
a radiation source for irradiating a patient; and
a magnetic resonance tomography apparatus, wherein the magnetic resonance tomography apparatus comprises a controller, a plurality of local coils for arrangement on the patient, and a plurality of transmission devices, the plurality of transmission devices being configured to supply the plurality of local coils with radiofrequency signals independently of one another,
wherein the controller is configured to actuate the plurality of transmission devices in such a way that a predetermined region in a body of the patient is heated to a predetermined temperature by means of the radiofrequency signals emitted by the plurality of local coils,
wherein the radiation source is configured to irradiate a target in the predetermined region.

2. The irradiation system as claimed in claim 1, wherein the magnetic resonance tomography apparatus is configured to register an increase in temperature in the predetermined region by means of a magnetic resonance measurement.

3. The irradiation system as claimed in claim 1, wherein the predetermined temperature amounts to a maximum of 42 degrees Celsius.

4. The irradiation system as claimed in claim 1, wherein each of the plurality of local coils is configured in such a way that the plurality of local coils are not mutually influenced by their signals.

5. The irradiation system as claimed in claim 1, wherein the controller and the plurality of transmission devices are configured to provide the radiofrequency signals of an individual local coil of the plurality of local coils with a phase angle that is predetermined in each case.

6. The irradiation system as claimed in claim 1, wherein the controller has a determination unit, the determination unit being configured to determine control signals for the plurality of transmission devices on the basis of a predetermined temperature distribution in the patient by means of which the predetermined temperature distribution in the patient is substantially achieved.

7. The irradiation system as claimed in claim 6, wherein the controller is configured to actuate the plurality of transmission devices in such a way that a deviation between the predetermined temperature distribution and the measured increase in temperature is reduced.

8. The irradiation system as claimed in claim 1, wherein the controller is configured either to actuate the plurality of transmission devices by means of a first control signal which is configured to heat the predetermined region in the body of the patient to the predetermined temperature by means of radiofrequency signals by means of the plurality of transmission devices and the plurality of local coils or to actuate the plurality of transmission devices by means of a second control signal which is configured to generate an imaging visualization by means of the magnetic resonance tomography apparatus.

9. The irradiation system as claimed in claim 8, wherein the controller is configured to actuate the plurality of transmission devices by means of the first control signal when the radiation source emits no radiation onto the patient.

10. A method for operating an irradiation system comprising a radiation source for irradiating a patient and a magnetic resonance tomography apparatus, the magnetic resonance tomography apparatus comprising a controller, a plurality of local coils for arrangement on the patient, and a plurality of transmission devices which are configured to supply the plurality of local coils with radiofrequency signals independently of one another, wherein the controller actuates the plurality of transmission devices in such a way that the radiofrequency signals emitted by the plurality of local coils heat a predetermined region in a body of the patient by a predetermined temperature, and wherein the radiation source irradiates a target in the predetermined region.

11. The method as claimed in claim 10, wherein, in a heating phase, the controller actuates the plurality of transmission devices by means of a first control signal which is configured to heat the predetermined region in the body of the patient to a predetermined temperature by means of the plurality of transmission devices and the plurality of local coils, wherein, in an irradiation phase in which the radiation source irradiates the target, the controller actuates the plurality of transmission devices by means of a second control signal which is configured to generate an imaging visualization by means of the magnetic resonance tomography apparatus and the magnetic resonance tomography apparatus generates the imaging visualization, and wherein the heating phase does not overlap with the irradiation phase.
